(19) United States
(12) Patent Application Publication
(10) Pub. No.: US 2025/0295844 A1
(43) Pub. Date: Sep. 25, 2025

(54) SYSTEM COMPRISING AN EXTRACORPOREAL BLOOD TREATMENT DEVICE AND A HEAT EXCHANGER

(71) Applicant: B. Braun Avitum AG, Melsungen (DE)

(72) Inventors: Waldemar Janik, Melsungen (DE); Joshua Lindner, Kassel (DE)

(21) Appl. No.: 19/083,952

(22) Filed: Mar. 19, 2025

(30) Foreign Application Priority Data
Mar. 25, 2024 (DE) 10 2024 108 454.5

(51) Int. Cl. A61M 1/16

(52) U.S. Cl. CPC A61M 1/1664; A61M 2205/3368; A61M 2205/3633

(57) ABSTRACT

A system includes an extracorporeal blood treatment device and a heat exchanger for heat exchange between dialysate flowing from the extracorporeal blood treatment device and permeate to be supplied to the extracorporeal blood treatment device. The extracorporeal blood treatment device and the heat exchanger are configured as separate devices.

### CROSS-REFERENCE TO RELATED APPLICATION

This application claims priority under 35 U.S.C. § 119 to German Application No. 2024 108 454.5, filed on Mar. 25, 2024, the content of which is incorporated by reference herein in its entirety.

### FIELD

The present disclosure relates to a system comprising an extracorporeal blood treatment device, for example a dialysis machine, and a heat exchanger for heat exchange between dialysate and permeate and a heat exchanger for use in such a system.

### BACKGROUND

In dialysis systems, the heat exchanger is usually integral with the extracorporeal blood treatment device, such as a dialysis machine, which in practice means that they are installed in a common fluid system and are often also enclosed in a common housing. However, there are very different application scenarios for such devices, for example dialysis machines, and the known solutions cannot always meet the desired application scenarios, which can result in reduced efficiency, for example.

One task underlying the present disclosure is to provide a system that is suitable for a wider range of application scenarios.

### SUMMARY

The present disclosure is directed to a system that includes an extracorporeal blood treatment device and a heat exchanger, and is also directed to a heat exchanger for use in such a system.

The system according to the present disclosure comprises an extracorporeal blood treatment device, in particular a dialysis machine, and a heat exchanger for exchanging heat between dialysate flowing from the extracorporeal blood treatment device and permeate to be supplied to the extracorporeal blood treatment device, wherein the extracorporeal blood treatment device and the heat exchanger are configured as separate devices.

In the following description, the features and explanations are described by way of example of a “dialysis machine” as a representative of the “extracorporeal blood treatment device”, which is intended to include all devices for extracorporeal blood treatment, for example devices for hemofiltration, hemodiafiltration, blood gassing or apheresis in addition to the dialysis machine.

The heat exchanger can be used in hemodialysis and/or peritoneal dialysis, for example.

In other words, a system is provided which comprises a heat exchanger external to the dialysis machine. The heat exchanger can be independent of the dialysis machine, in particular structurally, such that it can be connected to the dialysis machine and disconnected from the dialysis machine without modification to the dialysis machine and/or the heat exchanger and/or without opening the dialysis machine. The heat exchanger can be independent of the dialysis machine, in particular structurally, in such a way that it can be used simultaneously or successively with several dialysis machines without modifying the dialysis machine.

Permeate can be defined as the fresh solution in the direction of flow in normal operation upstream of the dialysis machine, in particular before concentrates are added to produce the dialysis fluid. Before reaching the dialysis machine, one or more concentrates are usually added to the permeate during normal operation, usually an alkaline and an acidic concentrate. The resulting mixture is called dialysis fluid or dialysis solution and is fed to the dialysis machine. The used solution after passing through the dialysis machine can be referred to as dialysate. In normal operation, the dialysate is usually warmer than the permeate and can preheat the permeate by means of the heat exchanger so that the heat from the dialysate can still be used to good effect.

Currently, the heat exchanger is configured integrally with the dialysis machine, which in practice means that they are installed in a common fluid system and often enclosed in a common housing.

The system according to the present disclosure deviates from this in that the dialysis machine and the heat exchanger are configured as separate devices.

This can enable the system to be highly efficient for different requirements. For example, the dimensioning of the heat exchanger can be selected according to demand. System components can be used flexibly to ensure optimum operation. For example, several heat exchangers can be provided for one dialysis machine or one heat exchanger for several dialysis machines. Multiple heat exchangers can be used independently or in series. Heat exchangers can also be easily added, removed or exchanged and/or the circuit of the components (heat exchanger and dialysis machine(s)) can be flexibly adapted to requirements. The increased flexibility means that a high level of efficiency can be guaranteed for a wide range of applications. A system can therefore be made available that is suitable for a wider range of application scenarios.

According to the present disclosure, the dialysis machine and the heat exchanger may be connected to each other via a first connector of the dialysis machine, a second connector of the dialysis machine, a first connector of the heat exchanger and a second connector of the heat exchanger. The system can optionally comprise a first line, for example a first hose, and a second line, for example a second hose. The first connector of the heat exchanger can be connected to the first connector of the dialysis machine by means of the first line and the second connector of the heat exchanger can be connected to the second connector of the dialysis machine by means of the second line. Optionally, the first line and/or the second line can be thermally insulated.

This means that the dialysis machine and the heat exchanger can be separably connected to each other via the respective connectors. The connection can be a direct connection, provided the connectors are compatible with each other, or an indirect connection, for example via an adapter or, as described above, via lines, wherein two connectors are connected to each other via a line. This allows the dialysis system to be configured easily and flexibly. By means of corresponding additional connectors on the dialysis machine and/or on the heat exchanger and/or by means of correspondingly configured line sections and/or switching elements, it is also possible to connect several dialysis machines to one heat exchanger and/or to connect several heat exchangers to one dialysis machine. In this way, a suitable configuration can be created, adapted to the respective usage scenario, which enables high efficiency for different usage scenarios. Optional thermal insulation of the lines makes it possible to achieve this flexibility and efficiency while keeping heat losses low, even with longer lines or greater distances.

The heat exchanger can comprise a heat transfer section, at least one valve that can be switched by means of an actuator and a sensor, in particular a temperature sensor. The heat exchanger can be configured to switch the valve by means of the actuator based on sensor data from the sensor. In particular, the heat exchanger can be configured to switch in such a way that when a first temperature threshold value of a fluid flowing from the dialysis machine through the heat exchanger, for example the dialysate or a fluid used during disinfection, decalcification and/or cleaning of the dialysis machine, is detected to be exceeded, the at least one valve is switched by means of the sensor in such a way that the outflowing fluid and/or the permeate does not flow through the heat transfer section.

The provision of such a valve can make it possible to divert the flow of permeate and/or the outflowing fluid/liquid in such a way that no heat exchange takes place between the permeate and the outflowing fluid. In particular, for example, the permeate can be diverted so that it does not flow through the heat transfer section.

The switching described above also enables, for example, heat utilization of the heat of the outflowing fluid for other processes. Coupling to the exceeding of a temperature threshold can be a safety feature that makes it possible to prevent the permeate from being heated too much by the outflowing fluid and/or can make it possible to use very high temperatures of the outflowing fluid favorably, for example for processes that require a higher temperature, such as cleaning processes.

The heat exchanger can comprise a/the temperature sensor and the heat exchanger can be configured to automatically supply a maintenance fluid, for example cleaning agent, disinfectant or descaling agent, into flow paths of the heat exchanger, in particular into a/the heat transfer section of the heat exchanger, when a second temperature threshold value of a fluid flowing from the dialysis machine through the heat exchanger, for example the dialysate or a fluid used for disinfection, descaling and/or cleaning of the dialysis machine, is exceeded.

Cleaning processes are generally more effective at higher temperatures. Recognizing and using a sufficiently high temperature of the draining fluid can be used as an alternative to dedicated heating for cleaning purposes or can support such heating. This in turn increases the efficiency of the system.

The heat exchanger can have a maintenance fluid supply section via which a/the maintenance fluid, for example cleaning agent, disinfectant or descaling agent, can be fed into flow paths of the heat exchanger, in particular into a/the heat transfer section of the heat exchanger

In particular, the system can be configured in such a way that the automatic supply of the cleaning fluid described above comprises an automatic establishing of a fluid connection between the maintenance fluid supply section and the flow paths, in particular the heat transfer section, when a second temperature threshold is exceeded. This can be different, in particular higher, than the first temperature threshold. For example, the system can be configured to automatically switch a valve to establish the fluid connection.

The system can comprise several dialysis machines and the heat exchanger can be connected to the dialysis machines via their respective first connector and second connector. For this purpose, the heat exchanger may comprise a plurality of first and second connectors, each of which is connected to the first and second connectors of the dialysis machines. Alternatively or additionally, a first and a second connector of the heat exchanger can each be connected to several first and second connectors of the dialysis machines. The system may comprise corresponding switching elements and/or line sections.

Such a connection allows several dialysis machines to share one heat exchanger. This enables better utilization of the heat exchanger and/or a more even supply to the dialysis machines. It can also enable a demand-oriented supply. This can increase overall efficiency. This also enables greater flexibility in terms of scaling the overall system.

The system can include several of the heat exchangers. This allows greater flexibility in terms of scaling the overall system. In addition, a degree of redundancy can be provided that allows failures due to malfunctions, cleaning or maintenance to be compensated for. In particular, the system can comprise several heat exchangers connected in series in relation to the direction of flow.

According to the present disclosure, the heat exchanger can be configured as a counterflow heat exchanger. This is particularly advantageous in connection with dialysis systems due to the usual configuration and operation.

According to the present disclosure, the heat exchanger can be configured as a double tube recuperator, tube bundle recuperator or plate recuperator.

The heat transfer section of the heat exchanger can be made of stainless steel or polymer-based material, in particular polypropylene or polyphenylene sulphide. Stainless steel has high thermal conductivity and robustness, whereas polymer-based material is lighter and therefore more transportable and can be formed in a variety of shapes, for example using additive manufacturing processes, for example to optimize the exchange surface and/or the flow behavior.

The heat exchanger can stand on the floor during normal operation. A self-standing heat exchanger can allow the heat exchanger to be dimensioned as required, which can be more difficult with a suspended configuration, for example. It is also not necessary to pay attention to compatibility with the configuration of the dialysis machine.

The heat exchanger can have rollers by means of which the heat exchanger can be transported, in particular on which the heat exchanger is supported during transportation and optionally during normal operation. This makes it possible to position the heat exchanger flexibly in a way that is suitable for operation, even with larger heat exchangers, which is particularly advantageous for flexible system configurations (e.g. connecting several heat exchangers together and/or connecting to one or more dialysis machines). Ease of positioning can also prevent heat loss, as the distance to the dialysis machine can be easily reduced by repositioning if appropriate.

The system can comprise a suspension system configured to suspend the heat exchanger from the dialysis machine, in particular from a machine housing of the dialysis machine.

This enables a short flow path between the dialysis machine and heat exchanger and therefore also lower heat losses. The mobility of the system can also be increased because it is easier to move them together. This means that some of the advantages of integrated heat exchangers can also be achieved for the external heat exchanger.

The heat exchanger can be arranged upstream of an inlet valve to the dialysis fluid circuit of the dialysis machine in relation to the permeate feed direction. This arrangement enables flexible connection to and disconnection from the dialysis machine and is therefore advantageous for a flexible configuration of the system, for example by easy replacement of the heat exchanger without interfering with the dialysis fluid circuit of the dialysis machine.

The dialysis fluid circuit can, for example, include the complete hydraulic system between the permeate inlet of the dialysis machine and the dialysate outlet. For example, the dialysis fluid circuit may comprise a water treatment section in the dialysis machine, a degassing section for degassing the water, a dialysis fluid treatment section, sections and/or feeds for conveying through the dialyzer, sections and/or devices for balancing dialysis fluid and dialysate and/or sections and devices for conveying to the drain. In particular, the dialysis fluid circuit may include the dialyzer, if one is fitted. The heat exchanger may thus be considered to be located upstream of the (internal) hydraulic system of the dialysis machine, for example.

The heat exchanger can be arranged between a ring main system and the dialysis machine with respect to the permeate feed direction, in particular where the system comprises a ring main system and the dialysis machine is connected to the ring main system via the heat exchanger during normal operation.

The ring main system can, for example, comprise a water line that comes from a water treatment system (e.g. reverse osmosis system) and supplies dialysis machines with fresh water during operation. Unused water can be fed back to the reverse osmosis system via the ring main system during operation.

The aforementioned arrangement of the heat exchanger allows a particularly high degree of flexibility with regard to the system configuration. If, for example, the heat exchanger were integrated into the ring main system, some of the above-mentioned flexible design and configuration options, particularly with regard to replacement, dimensioning and flexible wiring, might be more difficult to implement.

As explained above, the dialysis machine can have a machine housing. A first connector and a second connector of the dialysis machine can be arranged on the housing, in particular on the outside of the housing.

This configuration makes it possible to shield the dialysis machine and still provide a simple way of connecting heat exchangers to the dialysis machine fluidically. In particular, a heat exchanger can be flexibly connected without interfering with the dialysis fluid circuit of the dialysis machine, or more precisely without interfering with the dialysis machine.

The heat exchanger can have a housing. A first connector and a second connector of the heat exchanger can be arranged on the housing, in particular on the outside of the housing.

Such a housing enables shielding, in particular heat shielding, while at the same time making it easy to connect the heat exchanger flexibly.

The dialysis machine can have the machine housing and the heat exchanger can be arranged outside the machine housing. In particular, the entire heat exchanger can be arranged completely outside the machine housing.

The heat exchanger can have the housing described above and the dialysis machine can have the machine housing, wherein the housing of the heat exchanger is arranged outside the machine housing of the dialysis machine

The above features are particularly advantageous in terms of flexibility, as the dialysis machine and the heat exchanger can be shielded and moved independently of each other and the system can therefore be configured flexibly.

The heat exchanger can be connected to the dialysis machine in such a way that, during normal operation, a heat exchange takes place in the heat exchanger, in particular in a/the heat transfer section of the heat exchanger, between the permeate to be supplied to the dialysis machine by the heat exchanger and the dialysate flowing out of the dialysis machine. In particular, the heat exchanger can comprise corresponding fixed flow paths or flow paths that can be switched by means of valves. The heat exchange can be made possible by directing the flow of fluids into corresponding flow paths.

The system, in particular the heat exchanger, can have a display unit which is configured to display a valve position of a/the valve and/or sensor data of a/the sensor, in particular a temperature of a fluid flowing from the dialysis machine, in particular the dialysate. For example, a display unit, such as a screen, can be arranged on a housing of the heat exchanger or integrated into the housing of the heat exchanger. By means of such a display unit, the status of the system, in particular of the heat exchanger, can be output to a user, for example, which can also enable the user to intervene in the operation, for example.

The system, in particular the heat exchanger, can have a power supply, in particular a battery and/or mains power supply, for operating the sensor and/or for reading out the sensor and/or for operating the display unit and/or for controlling an actuator for switching the valve and/or for operating the actuator.

Alternatively or additionally, the heat exchanger can have a thermoelectric generator which is configured to provide energy for operating the sensor and/or for reading out the sensor and/or for operating the display unit and/or for controlling an actuator for switching the valve and/or for operating the actuator and/or for charging the battery by means of the temperature difference between a fluid flowing from the dialysis machine, in particular the dialysate, and permeate. The generator can make use of the Seebeck or Peltier effect.

This enables autonomous operation of the heat exchanger, at least temporarily. It can also allow excess heat to be put to good use. This can further increase efficiency.

The system can have a data connection between the heat exchanger and the dialysis machine and can be configured to transmit sensor data, in particular data from a temperature sensor, from the heat exchanger to the dialysis machine, in particular to regulate the dialysis fluid temperature.

Optionally, the data connection and the power supply can be provided by means of a common connecting element, for example by means of a connecting cable that allows power supply and data transfer.

Dialysis machines usually contain one or more temperature sensors whose measured values are used to control the operation of the dialysis machine. This control is improved if temperature data from the heat exchanger is also taken into account. For example, during the production of dialysis fluid, which takes place inside a dialysis machine, not only the composition of the fluid but also its temperature can be adjusted. Temperature control is often a complex process. This is particularly due to the fact that the controlled system is usually quite large, i.e. there may be many other components such as valves, chambers, branches, filters and sensors between the heating device and the location where the target temperature is to be achieved. In most cases, there are several temperature sensors distributed along the control path, the data from which are used to control the dialysis fluid temperature. According to the present disclosure, the temperature sensor data from the heat exchanger can also be taken into account in this control.

Data provided by the dialysis machine to the heat exchanger can be used to switch valves of the heat exchanger, for example for the valve described above, which can be switched so that the permeate and/or dialysate does not flow through the heat transfer section. For example, if the target temperature (setpoint temperature) is lower than the current temperature of the dialysis fluid, it may be necessary to switch off a heater for cooling and convey the warm dialysate into the drain. In such a case, the permeate could be heated unintentionally in the heat exchanger. By exchanging information on the target temperature from the dialysis machine to the heat exchanger, the valve of the heat exchanger can be opened, for example as described above, to prevent heat exchange. The cooling of the dialysis machine's hydraulics can thus be made more efficient.

The present disclosure also relates to a method for exchanging heat between dialysate flowing from a dialysis machine and permeate to be supplied to the dialysis machine by means of a heat exchanger, wherein the dialysis machine and the heat exchanger are configured as separate devices of a system, in particular the system of the present disclosure. The present disclosure also relates to a use of the system according to the present disclosure for exchanging heat between dialysate flowing from the dialysis machine and permeate to be supplied to the dialysis machine. The advantages and features described above apply analogously.

The present disclosure also relates to a heat exchanger for use in a system according to the present disclosure, in particular for connection to one or more dialysis machines according to the present disclosure.

In particular, the heat exchanger can be configured as described above in connection with the system.

### BRIEF DESCRIPTION OF THE DRAWINGS

Further examples and embodiments are explained below with reference to the figures.

FIG. 1 shows a schematic and not-to-scale representation of a system according to the present disclosure.

FIG. 2 shows a schematic and not-to-scale representation of a system according to the present disclosure.

FIG. 3 shows a schematic and not-to-scale representation of a heat exchanger of a system according to the present disclosure.

FIG. 4 shows a schematic and not-to-scale representation of a heat exchanger of a system according to the present disclosure.

### DETAILED DESCRIPTION

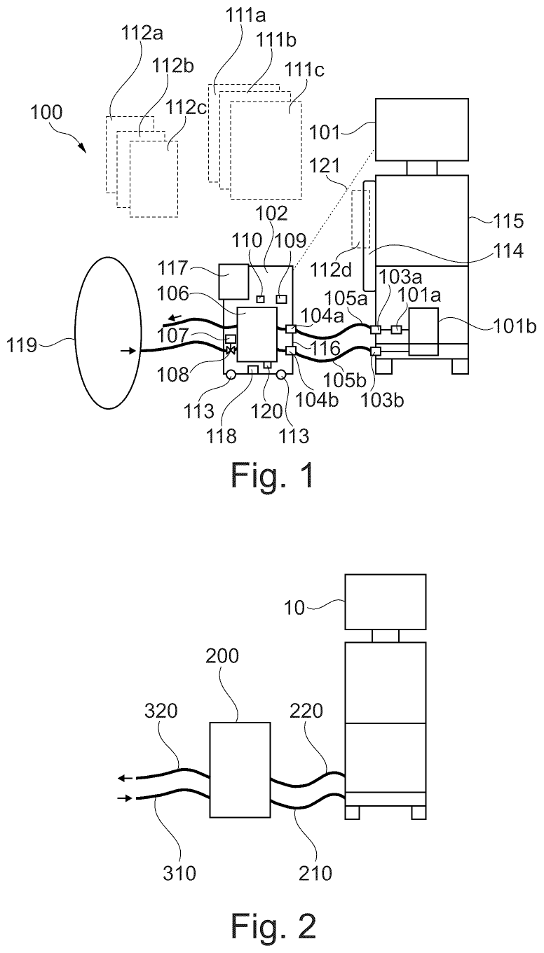

FIG. 1 shows a system 100 comprising an extracorporeal blood treatment device 101, for example a dialysis machine, and a heat exchanger 102 for exchanging heat between the dialysate flowing from the dialysis machine and the permeate to be supplied to the dialysis machine. The dialysis machine and the heat exchanger are configured as separate devices. For example, the heat exchanger may be a counterflow heat exchanger. Various types of heat exchangers are possible, for example a double tube recuperator, a tube bundle recuperator or a plate recuperator.

The heat exchanger can be connected to the dialysis machine in such a way that, during normal operation, a heat exchange takes place in the heat exchanger, in particular in a heat transfer section of the heat exchanger, between the permeate to be supplied to the dialysis machine by the heat exchanger and the dialysate flowing out of the dialysis machine.

A first connector 103a and a second connector 103b of the dialysis machine and a first connector 104a and 104b of the heat exchanger are shown as examples. The heat exchanger and the dialysis machine are connected to each other via the connectors. More precisely, the first connectors 103a and 104a can be connected to each other by means of a first line 105a, which can be in the form of a hose, for example. The second connectors 103b and 104b can be connected to each other by means of a second line 105b, which can be configured as a hose, for example. The lines can optionally be thermally insulated.

The heat exchanger can, for example, be arranged upstream of an inlet valve 101a to the dialysis fluid circuit 101b of the dialysis machine with respect to the permeate feed direction.

FIG. 1 shows an exemplary heat transfer section 106 of the heat exchanger. This can be made of stainless steel or polymer-based material, in particular polypropylene or polyphenylene sulphide.

Optionally, as shown in FIG. 1, an actuator 107, a valve 108 that can be switched by the actuator and a sensor 109 can be provided, which can be part of the heat exchanger, for example. The sensor can be a temperature sensor, for example.

The heat exchanger can, for example, be configured to switch the valve by means of the actuator based on sensor data from the sensor, in particular to switch in such a way that when a first temperature threshold value of the dialysate is detected as being exceeded by the sensor, the valve is switched in such a way that the permeate does not flow through the heat transfer section. This means that the permeate is no longer heated further by means of heat transfer if the temperature of the dialysate is too high. Alternatively, for example, the permeate can always flow through the heat transfer section and the dialysate can be diverted past this section as required

The heat exchanger can be configured to automatically feed a maintenance fluid, for example cleaning agent, disinfectant or descaling agent, into flow paths of the heat exchanger, in particular into the heat transfer section 106 of the heat exchanger, when a second temperature threshold value of the dialysate is exceeded. This can be done, for example, using measured values from sensor 109.

The heat exchanger can optionally have a maintenance fluid supply section 110, via which maintenance fluid, for example cleaning agent, disinfectant or descaling agent, can be supplied into flow paths of the heat exchanger, in particular into the heat transfer section 106 of the heat exchanger.

The system can optionally comprise several dialysis machines. In FIG. 1, further optional dialysis machines 111a to 111c are shown as examples in dashed lines. The heat exchanger is connected to the dialysis machines via their respective first connector and second connector. The fact that three further dialysis machines are shown here is to be understood as purely exemplary; fewer or more dialysis machines may also be provided.

The system can (alternatively or in addition to any additional dialysis machines) comprise additional heat exchangers 112a to 112d, which are shown in FIG. 1 as examples in dashed lines. These can each be configured like the heat exchanger 102. In particular, at least some of the heat exchangers can be connected in series with respect to the direction of flow. The fact that four further heat exchangers are shown here is to be understood as purely exemplary; fewer or more heat exchangers may also be provided.

The heat exchanger can stand on the floor during normal operation, as shown in FIG. 1 for the heat exchanger 102. The heat exchanger can optionally have rollers 113 by means of which the heat exchanger can be transported, in particular on which the heat exchanger is supported during transportation and optionally during normal operation.

As shown on the optional heat exchanger 112d, the heat exchanger can also be suspended from the dialysis machine. For this purpose, the system can have a suspension system 114 by means of which the heat exchanger is suspended from the dialysis machine. In particular, the heat exchanger can be suspended from a machine housing 115 of the dialysis machine.

The first and second connectors 103a and 103b of the dialysis machine can be arranged on the outside of the machine housing. The first and second connectors 104a and 104b of the heat exchanger can be arranged on the outside of a housing 116 of the heat exchanger.

In FIG. 1, the entire heat exchanger, in particular including the housing 116, is shown arranged completely outside the machine housing 115 of the dialysis machine. This is an exemplary embodiment of the feature that the dialysis machine and the heat exchanger are separate devices.

The system, in particular the heat exchanger, can have a display unit 117 which is configured to display a valve position of the valve 108 and/or sensor data of the sensor 109, in particular a temperature of the dialysate.

The system, in particular the heat exchanger, can have a power supply 118, in particular a battery and/or mains power supply, for operating the sensor and/or for reading out the sensor and/or for operating the display unit and/or for controlling the actuator 107 for switching the valve and/or for operating the actuator.

The heat exchanger can have a thermoelectric generator 120, which is configured to use the temperature difference between the dialysate and permeate to provide energy for operating the sensor and/or for reading out the sensor and/or for operating the display unit and/or for controlling an actuator for switching the valve and/or for operating the actuator and/or for charging the battery.

The system can optionally have a data connection 121 between the heat exchanger and the dialysis machine and can be configured to transmit sensor data, in particular data from the temperature sensor, from the heat exchanger to the dialysis machine, in particular for controlling the permeate temperature.

FIG. 1 also shows a ring main system 119, which can optionally be part of the system of the present disclosure. The heat exchanger may be arranged between the ring main system and the dialysis machine with respect to the permeate feed direction. In particular, the dialysis machine can be connected to the ring main system via the heat exchanger during normal operation. The ring main system comprises, for example, a water line that comes from a water treatment system (e.g. reverse osmosis system) and supplies dialysis machines with fresh water.

Further features and benefits are described below.

The present disclosure relates to a system comprising a dialysis machine and a heat exchanger, which is also referred to below as a recuperator. These are configured as independent devices. Therefore, the recuperator is also referred to as an external recuperator. The heat exchanger is intended for a dialysis machine, i.e. an extracorporeal blood treatment device, as an external recuperator that can be installed between the ring main system and the device, in particular as a retrofit, in order to use the waste heat from outflowing fluids to heat inflowing fluids and thus save energy.

In particular, the heat exchanger of the present disclosure may not be a component of the dialysis machine, a component of the ring main system or a component of the reverse osmosis system.

Recuperators and their use in dialysis machines for preheating high-purity dialysis water (permeate) are well known. The recuperator is part of the machine and is installed in the hydraulic system. In the simplest case, it is a heating coil through which the outflowing dialysate flows and which is located in a flow tank. The permeate to be heated flows into the flow tank and is subsequently mixed with an alkaline and an acidic component. However, simple heating coils have the disadvantage that they are not very efficient and not too much heat can be recovered. Alternatively, plate recuperators are also used, which are associated with a larger exchange surface and therefore increase the degree of efficiency. The disadvantage, however, is that plate recuperators require a lot of maintenance and are difficult or almost impossible to empty when installed, although this must be done before a dialysis machine is delivered. In some cases, heat exchangers (which may be removable) are part of the dialysis machine. However, the dialysis machine must be opened for removal, which can only be carried out by service technicians. In general, the efficiency increases with increasing exchange surface area. However, as the installation space within a dialysis machine is limited, the waste heat cannot always be used efficiently.

If an attempt were made to configure the recuperator as part of the ring main system instead, integrating the recuperator into an existing ring main system would involve a great deal of effort or would not be possible at all.

The system of the present disclosure makes it possible to provide a recuperator that uses the waste heat from the outflowing dialysate to preheat the inflowing permeate. The recuperator can be integrated between the ring main system and the dialysis machine. The recuperator is therefore not part of the machine. Likewise, the recuperator is optionally not part of the ring main system. The configuration, which is independent of the dialysis machine and possibly the ring main system, makes it easier to retrofit existing machines with this external recuperator.

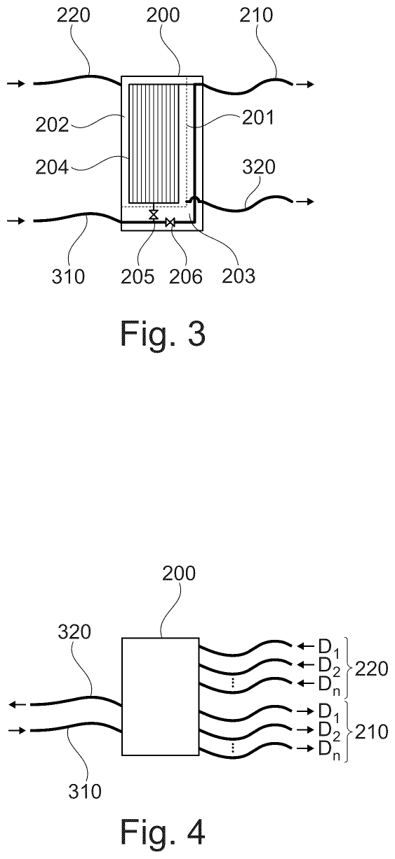

This means that the heat exchanger can be used as required without having to make any changes to the machine or, if appropriate, to the ring main system. Several machines can be connected to one heat exchanger (FIG. 4) and several heat exchangers in series and/or parallel are possible. There is the option of a passive configuration with continuous heat transfer or an active configuration in which the valve positions in the heat exchanger are actively adjusted depending on the temperature, for example paths are released (FIG. 3).

FIG. 2 schematically shows a dialysis machine 10 according to the present disclosure, to which a recuperator 200 is connected. Permeate from a ring main system or from a reverse osmosis system flows through line 310 into the recuperator 200 and then through line 210 further into the dialysis machine 10. The (warm) dialysate flows through line 220 into the recuperator 200 and then through line 320 into the drain or treatment unit. The fluids are led past each other in opposite directions (counterflow principle). The heat exchange between the dialysate and permeate takes place in the recuperator 200. Various types of recuperator 200 are possible. These include, in particular, double tube, tube bundle and plate recuperators. Since the recuperator 200 is located outside the dialysis machine 10, it is not absolutely necessary to pay attention to the size limit, as there is sufficient space outside the machine. Stainless steel, which has good thermal conductivity and high robustness, can be used as a corrosion-resistant material for the heat transfer unit. Alternatively, polymer-based units are conceivable. They offer the advantage that almost any structure can be implemented, particularly using additive manufacturing processes, and the exchange surface and flow behavior can therefore be optimized. Possible materials include thermally conductive polypropylene or polyphenylene sulphide. Due to their lower weight compared to stainless steel, polymer-based recuperators can also be transported more easily. The recuperator 200 can be placed on the floor or have rollers so that it can be moved if appropriate. It is also possible to equip the recuperator with an element for hanging on a dialysis machine 10. It is possible to keep the distance between recuperator 200 and dialysis machine 10, and thus the length of lines 210 and 220, as short as possible in order to avoid heat loss. The lines 210 and 220 can also be thermally insulated. For cleaning or disinfection or decalcification, the heat transfer unit may have a point for applying a cleaning agent, which then flushes the flow paths. The present disclosure also includes the possibility of connecting several recuperators in series in order to increase efficiency.

It is possible to equip the recuperator with further elements, i.e. in particular sensors and actuators, in addition to the actual heat transfer unit. FIG. 3 shows an example of the recuperator 200 from FIG. 2 in such an embodiment. A wall 201 divides the interior of the recuperator 200 into two spaces 202 and 203, with the actual heat transfer unit 204 being located in space 202. The valves 205 and 206, through which the permeate to be heated enters either into the heat transfer unit 204 or directly into line 210 from line 310, are located in chamber 203. The latter avoids heating the permeate, which is particularly advantageous after disinfection, since hot fluid flows out through line 220 during disinfection and fresh permeate would only be heated unnecessarily (above a physiologically tolerable temperature) in some cases (for example, when a new dialysis therapy is to be prepared). The recuperator 200 can also have at least one temperature sensor for this purpose, which measures at least the temperature of the dialysate flowing from the dialysis machine 10. If a threshold value (e.g. 40° C.) is exceeded, the valve 205 can close and the valve 206 can open in order to counteract undesired heating of the permeate. The temperature can also be used to control the automatic application of a cleaning agent into the flow paths of the recuperator depending on the temperature, as the cleaning efficiency increases as the temperature rises.

The recuperator 200 can also have a display unit that shows, for example, the valve position and/or temperatures and/or other sensor data. The energy supply for this can be provided by a battery or from the mains. It is also possible to make the recuperator energy self-sufficient. For example, the Seebeck or Peltier effect can be used to generate a voltage based on the temperature difference between the warm dialysate and the colder permeate, which is then used to read out sensor values, control actuators and/or operate the display unit. Furthermore, a wireless or wired connection to a dialysis machine is possible. It is conceivable, for example, to transmit the temperature data of the recuperator to the connected machine in order to use this data to optimize the control of the dialysis fluid temperature. The present disclosure optionally provides for the use of one recuperator for several machines. For this purpose, the recuperator 200 has a plurality of permeate lines 210 leading to the dialysis machines D₁ to Dₙ and dialysate lines 220 leading from the machines D₁ to Dₙ to the recuperator 200 (see, for example, FIG. 4).

This recuperator can also be retrofitted between a conventional ring main system and dialysis machines.

Although the present disclosure is illustrated and described in detail in the drawings and the foregoing description, these illustrations and descriptions are to be considered exemplary and not limiting. The present disclosure is not limited to the disclosed embodiments. In view of the foregoing description and drawings, it will be apparent to those skilled in the art that various modifications can be made within the scope of the present disclosure.

1. A system comprising:
an extracorporeal blood treatment device; and
at least one heat exchanger for heat exchange between dialysate flowing from the extracorporeal blood treatment device and a permeate to be supplied to the extracorporeal blood treatment device,
the extracorporeal blood treatment device and the at least one heat exchanger being separate devices.

2. The system according to claim 1, wherein:
the extracorporeal blood treatment device and the at least one heat exchanger are connected or connectable to each other via a first connector of the extracorporeal blood treatment device, a second connector of the extracorporeal blood treatment device, a first connector of the at least one heat exchanger and a second connector of the at least one heat exchanger,
the system further comprises a first line and a second line,
the first connector of the at least one heat exchanger is connected by the first line to the first connector of the extracorporeal blood treatment device, and
the second connector of the at least one heat exchanger is connected by the second line to the second connector of the extracorporeal blood treatment device.

3. The system according to claim 2, wherein the first line comprises a first hose, and the second line comprises a second hose.

4. The system according to claim 2, wherein the first line and/or the second line is/are thermally insulated.

5. The system according to claim 1, wherein the extracorporeal blood treatment device and the at least one heat exchanger are connected or connectable to each other via a first connector of the extracorporeal blood treatment device, a second connector of the extracorporeal blood treatment device, a first connector of the at least one heat exchanger and a second connector of the at least one heat exchanger.

6. The system according to claim 1, wherein:
the at least one heat exchanger comprises a heat transfer section, at least one valve switchable by an actuator, and a sensor, and
the at least one heat exchanger is configured to switch the at least one valve via the actuator based on sensor data from the sensor.

7. The system according to claim 6, wherein the sensor is a temperature sensor.

8. The system according to claim 7, wherein the at least one heat exchanger is configured to switch the at least one valve via the actuator in such a way that when a first temperature threshold value of a fluid flowing from the extracorporeal blood treatment device through the at least one heat exchanger is exceeded, the at least one valve is switched by the sensor in such a way that the permeate and/or the fluid does not flow through the heat transfer section.

9. The system according to claim 8, wherein the at least one heat exchanger is configured to automatically feed a maintenance fluid into flow paths of the at least one heat exchanger when a second temperature threshold value of a fluid flowing from the extracorporeal blood treatment device through the at least one heat exchanger is exceeded.

10. The system according to claim 9, wherein the maintenance fluid is a cleaning agent, a disinfectant or a decalcifying agent.

11. The system according to claim 9, wherein the flow paths of the heat exchange are in a heat transfer section of the at least one heat exchanger.

12. The system according to claim 1, wherein:
the at least one heat exchanger comprises a maintenance fluid supply section, and
a maintenance fluid is supplied into flow paths of the at least one heat exchanger via the maintenance fluid supply section.

13. The system according to claim 1, wherein:
the extracorporeal blood treatment device comprises a plurality of extracorporeal blood treatment devices,
each of the plurality of extracorporeal blood treatment devices comprises a first connector and a second connector, and
the at least one heat exchanger is connected to the plurality of extracorporeal blood treatment devices via the first connectors and the second connectors.

14. The system according to claim 1, wherein:
the at least one heat exchanger is configured as a counterflow heat exchanger, and/or
the at least one heat exchanger is configured as a double tube recuperator, a tube bundle recuperator or a plate recuperator.

15. The system according to claim 1, wherein at least one of:
the at least one heat exchanger stands on a floor during normal operation,
the at least one heat exchanger comprises rollers by which the at least one heat exchanger is transportable, and
the system comprises a suspension system adapted to suspend the at least one heat exchanger from the extracorporeal blood treatment device.

16. The system according to claim 1, wherein:
the at least one heat exchanger is arranged upstream of an inlet valve to a dialysis fluid circuit of the extracorporeal blood treatment device with respect to a permeate feed direction, and/or
the at least one heat exchanger is arranged between a ring main system and the extracorporeal blood treatment device with respect to the permeate feed direction.

17. The system according to claim 1, wherein:
the extracorporeal blood treatment device comprises a machine housing, and/or
the at least one heat exchanger has a housing.

18. The system according to claim 17, wherein:
the extracorporeal blood treatment device comprises the machine housing and the at least one heat exchanger is arranged outside the machine housing, and/or
the at least one heat exchanger comprises the housing and the extracorporeal blood treatment device comprises the machine housing, wherein the housing of the at least one heat exchanger is arranged outside the machine housing of the extracorporeal blood treatment device.

19. The system according to claim 1, wherein the at least one heat exchanger is connected to the extracorporeal blood treatment device in such a way that during normal operation in the at least one heat exchanger, a heat exchange takes place between permeate to be provided from the at least one heat exchanger to the extracorporeal blood treatment device and dialysate flowing out of the extracorporeal blood treatment device.

20. The system according to claim 1, wherein:
the system has a display unit configured to display a valve position of a valve and/or sensor data of a sensor, and/or
the system has a power supply for operating a sensor and/or for reading out the sensor and/or for operating the display unit and/or for controlling an actuator for switching a valve and/or for operating the actuator, and/or
the at least one heat exchanger has a thermoelectric generator configured to provide energy for operating a sensor and/or for reading out the sensor and/or for operating the display unit and/or for controlling an actuator for switching a valve and/or for operating an actuator and/or for charging a battery by a temperature difference between a fluid flowing from the extracorporeal blood treatment device and the permeate.

21. The system according to claim 1, further comprising a data connection between the at least one heat exchanger and the extracorporeal blood treatment device, the system configured to transmit sensor data from the at least one heat exchanger to the extracorporeal blood treatment device.
